(19) United States
(12) Patent Application Publication
(10) Pub. No.: US 2026/0367034 A1
(43) Pub. Date: Nov. 19, 2026

(54) Determining Chemical Concentration Through Polarization Resistance

(71) Applicant: ChampionX LLC, Sugar Land, TX (US)

(72) Inventor: Jeremy Moloney, Katy, TX (US)

(21) Appl. No.: 19/676,995

(22) Filed: May 14, 2026

Related U.S. Application Data
(60) Provisional application No. 63/807,124, filed on May 16, 2025.

(51) Int. Cl. G01N 27/416; G01N 33/28

(52) U.S. Cl. CPC G01N 27/4163; G01N 33/28

(57) ABSTRACT

Calibration polarization resistance curves are generated from calibration fluids, and concentration/polarization resistance data point pairs corresponding to a calibration time period from the curves are used to generate a calibration equation. For testing of chemical concentration in a sample of a fluid of a field system, a sample polarization resistance value is determined for the sample for a field time period that corresponds to the calibration time period of the calibration equation, and the calibration equation uses the sample polarization resistance value to calculate the concentration of the chemical in the fluid in the field system.

### CROSS REFERENCE TO RELATED APPLICATIONS

This application is a non-provisional patent application claiming the benefit of, and priority to, U.S. Provisional Patent Application No. 63/807,124, filed May 16, 2025, which is incorporated by reference herein in its entirety.

### FIELD OF THE DISCLOSURE

The present disclosure generally relates to determining the concentration of a chemical in a fluid, and more particularly to the use of polarization resistance determined for the fluid to determine the concentration of the chemical in the fluid.

### BACKGROUND

Chemical additives are added to fluids in various industries for various purposes. For example, some industries involve the production and/or transport of fluids that contain water or water vapor in a field system, such as an oil and gas system, a geothermal system, a carbon capture system, a carbon dioxide storage system, a carbon dioxide transport system, a hydrogen transport system, a water transport system, or a water treatment system. Chemical additives such as corrosion inhibitors, scale inhibitors, gas hydrate inhibitors, and biocides may be added to the fluids in the field systems to prevent corrosion on equipment surfaces, prevent scale formation on equipment surfaces, prevent blockages in natural gas pipelines due to gas hydrate formation, or to kill microorganisms in the fluid.

When determining the type and amount of chemical additive to introduce into the fluid(s) of such systems, laboratory tests may be performed under simulated field conditions. The chemical additive is then introduced into the field system based on the type and amount of chemical additive determined under the simulated field conditions in the laboratory tests. The rate of chemical introduction into the field system is initially based on the laboratory tests. Because of the differences between the laboratory and the field environments, even with laboratory testing results, the rate of chemical addition may need to be optimized in the field system. One way to optimize chemical addition in the field system is to monitor the concentration of the chemical additive at one more locations on the field system and adjust the rate of chemical additive addition based on the monitoring results.

Monitoring can include analysis of samples from the field system in the field by calorimetric techniques which are relatively quick to perform but are subject to inaccuracy. Monitoring can also be accomplished in by testing sample from the field system in a laboratory setting by liquid chromatography-mass spectrometry (LCMS) techniques, which are more accurate than calorimetric techniques, but are expensive, require expert scientific analysis, and can take long periods of time to get results.

One technique for monitoring corrosion inhibitor concentration includes generating a standard potentiodynamic polarization curve for a standard composition and a potentiodynamic polarization curve for a test composition, and comparing the curves to determine a concentration of the corrosion inhibitor in the test composition. However, simply comparing curves means that the test composition is tested for the same amount of time as the standard composition so as to accurately compare curves to determine concentration-causing a sample testing time of multiple hours (in addition to the time it takes for the lab to obtain the sample from the field) for a potentiodynamic polarization curve of the test composition to show a signature that would match a standard potentiodynamic polarization curve.

### SUMMARY

A method can include: adding a chemical to each of a plurality of calibration fluids at a test reference point in time for each of the plurality of calibration fluids, wherein a concentration of the chemical in each of the plurality of calibration fluids is known and is different in each of the plurality of calibration fluids; generating a polarization resistance curve versus time for each of the plurality of calibration fluids, wherein the polarization resistance curve for each of the plurality of calibration fluids starts at a common start time relative to the test reference point in time; and generating a calibration equation based on a calibration polarization resistance value in each of the polarization resistance curves at a calibration point in time that occurs after the test reference point in time; wherein the calibration equation calculates a concentration of the chemical in a sample of a field fluid when a sample polarization resistance value that was determined for the sample of the field fluid is input to the calibration equation.

An apparatus can include: a test vessel; an electrochemical probe coupled to the test vessel; a potentiostat; and a computer operably connected to the electrochemical probe, wherein the computer has one or more processors and a memory having instructions stored thereon that cause the one or more processors to: generate a polarization resistance curve versus time for each of a plurality of calibration fluids, wherein the polarization resistance curve for each of the plurality of calibration fluids starts at a common start time relative to a test reference point in time; and generate a calibration equation based on a calibration polarization resistance value in each of the polarization resistance curves at a calibration point in time that occurs after the test reference point in time; wherein the calibration equation calculates a concentration of a chemical in a sample of a field fluid when a sample polarization resistance value that was determined for the sample of the field fluid is input to the calibration equation.

Another method can include: determining a reference point in time at which a chemical was introduced into a field fluid; determining a sample polarization resistance value in a sample of the field fluid at a measurement point in time; inputting the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and determining, based on the calibration equation, the concentration of the chemical in the sample of the field fluid.

Another apparatus can include: a test vessel; an electrochemical probe coupled to the test vessel; a potentiostat; and a computer operably connected to the electrochemical probe, wherein the computer has one or more processors and a memory having instructions stored thereon that cause the one or more processors to: determine a reference point in time at which a chemical was introduced into a field fluid; determine a sample polarization resistance value in a sample of the field fluid at a measurement point in time; input the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and determine, based on the calibration equation, the concentration of the chemical in the sample of the field fluid.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates a graph of polarization resistance curves for the calibration fluids in the examples.

FIG. 2 illustrates a graph of chemical concentration versus polarization resistance, where the polarization resistance values are taken from the curves in FIG. 1 at a time of 4 hours in FIG. 1.

FIG. 3 illustrates a graph of chemical concentration versus polarization resistance, where the polarization resistance values are taken from the curves in FIG. 1 at a time of 7 hours in FIG. 1.

FIG. 4 illustrates a graph of chemical concentration versus polarization resistance, where the polarization resistance values are taken from the curves in FIG. 1 at a time of 11 hours in FIG. 1.

FIG. 5 illustrates a graph of polarization resistance curves for the test liquid in the examples.

### DETAILED DESCRIPTION

Concentrations disclosed herein are on weight basis unless otherwise specified. For example, 10 ppm of chemical in a fluid means parts-per-million based on a total weight of the fluid.

Disclosed are methods and apparatus for determining a concentration of a chemical in a fluid based on polarization resistance of the fluid. The methods and apparatus generate calibration polarization resistance curves from which a calibration equation is generated for data points having concentration and polarization resistance value pairs, for a common time period, as explained herein. For testing of chemical concentration in a field system, the methods and apparatus can measure a single polarization resistance value in a sample from the field system at a time period relative to a reference time that is the same time period for data points having concentration and polarization resistance value pairs that are used to generate the calibration equation. Using the calibration equations eliminates the need to perform a time-intensive experiment to generate a polarization resistance curve for the field sample, since only a single value is determined. Because no polarization resistance curve is generated for the field sample, the need to compare the curve to standard curves is avoided, and thus, the methods and apparatus disclosed herein determine the concentration of the chemical in the field system faster than techniques which compare sample curves to standard curves.

The fluid in the field system can be in liquid phase, in gas phase, or a mixture in gas phase and liquid phase. In some aspects, a liquid phase can be a water or brine or a liquid containing a water phase (water or brine) and a hydrocarbon phase (e.g., any one or mixture of liquid phase hydrocarbons). In some aspects, the gas phase can be water vapor or gaseous hydrocarbon(s) containing water vapor. The fluid can be contained in an oil and gas system, a geothermal system, a carbon capture system, a carbon storage system, a hydrogen transport system, a water transport system, or a water treatment system. For example, within one of these systems, the fluid can be contained in a pipeline, a process flow line, a valve, a separation vessel, a storage vessel, or a combination thereof.

While the fluid which can be tested according to this disclosure is not limited to the oil and gas industry, such fluids in the oil and gas industry may contain a chemical such as a corrosion inhibitor, a scale inhibitor, an asphaltene inhibitor, a gas hydrate inhibitor, or a biocide. These chemicals are common additives present in oil and gas fluids, for which the concentrations thereof are desired to be monitored.

Examples of corrosion inhibitors can include, but are not limited to, a quaternary ammonium compound, an imidazoline derivative, an organic sulfur compound, or combinations thereof. Quaternary ammonium compounds can include tetramethyl ammonium chloride, tetraethyl ammonium chloride, tetrapropyl ammonium chloride, tetrabutyl ammonium chloride, tetrahexyl ammonium chloride, tetraoctyl ammonium chloride, benzyltrimethyl ammonium chloride, benzyltriethyl ammonium chloride, phenyltrimethyl ammonium chloride, phenyltriethyl ammonium chloride, cetyl benzyldimethyl ammonium chloride, hexadecyl trimethyl ammonium chloride, dimethyl alkyl benzyl quaternary ammonium compounds, monomethyl dialkyl benzyl quaternary ammonium compounds, trimethyl benzyl quaternary ammonium compounds, trialkyl benzyl quaternary ammonium compounds, or combinations thereof. Imidazoline derivatives can be selected from an imidazoline derived from a polyamine and a long chain fatty acid. Examples of the polyamine can include ethylene diamine (EDA), diethylene triamine (DETA), triethylene tetraamine (TETA), or combinations thereof. Organic sulfur compounds can include a thiol (also known as a mercaptan), an organic disulfide, or combinations thereof.

Examples of scales inhibitors can include, but are not limited to, phosphates, phosphate esters, phosphoric acids, phosphonates, phosphonic acids, polyacrylamides, salts of acrylamido-methyl propane sulfonate/acrylic acid copolymer (AMPS/AA), phosphinated maleic copolymer (PHOS/MA), salts of a polymaleic acid/acrylic acid/acrylamido-methyl propane sulfonate terpolymer (PMA/AMPS), or combinations thereof.

Examples of asphaltene inhibitors include, but are not limited to, aliphatic sulphonic acids; alkyl aryl sulphonic acids; aryl sulfonates; lignosulfonates; alkylphenol/aldehyde resins and similar sulfonated resins; polyolefin esters; polyolefin imides; polyolefin esters with alkyl, alkylenephenyl or alkylenepyridyl functional groups; polyolefin amides; polyolefin amides with alkyl, alkylenephenyl or alkylenepyridyl functional groups; polyolefin imides with alkyl, alkylenephenyl or alkylenepyridyl functional groups; alkenyl/vinyl pyrrolidone copolymers; graft polymers of polyolefins with maleic anhydride or vinyl imidazole; hyperbranched polyester amides; polyalkoxylated asphaltenes, amphoteric fatty acids; salts of alkyl succinates; sorbitan monooleate; and polyisobutylene succinic anhydride, or combinations thereof.

Examples of gas hydrate inhibitors include, but are not limited to, thermodynamic hydrate inhibitors (THI), kinetic hydrate inhibitors (KHI), anti-agglomerates (AA), or combinations thereof.

Examples of thermodynamic hydrate inhibitors include, but are not limited to, NaCl salt, KCl salt, CaCl₂) salt, MgCl₂ salt, NaBr₂ salt, formate brines (e.g. potassium formate), polyols (e.g., glucose, sucrose, fructose, maltose, lactose, gluconate, monoethylene glycol, diethylene glycol, triethylene glycol, mono-propylene glycol, dipropylene glycol, tripropylene glycols, tetrapropylene glycol, monobutylene glycol, dibutylene glycol, tributylene glycol, glycerol, diglycerol, triglycerol, sugar alcohols (e.g. sorbitol, mannitol), or combinations thereof), methanol, propanol, ethanol, glycol ethers (e.g., diethyleneglycol monomethylether, ethyleneglycol monobutylether, or combinations thereof), alkyl or cyclic esters of alcohols (e.g., ethyl lactate, butyl lactate, methylethyl benzoate, or combinations thereof), or combinations thereof.

Examples of kinetic hydrate inhibitors and anti-agglomerates include, but are not limited to, polymers and copolymers, polysaccharides (e.g., hydroxy-ethylcellulose (HEC), carboxymethylcellulose (CMC), starch, starch derivatives, xanthan, or combinations thereof), lactams (e.g., polyvinylcaprolactam, polyvinyl lactam), pyrrolidones (e.g., polyvinyl pyrrolidone of various molecular weights), surfactants (e.g., fatty acid salts, ethoxylated alcohols, propoxylated alcohols, sorbitan esters, ethoxylated sorbitan esters, polyglycerol esters of fatty acids, alkyl glucosides, alkyl polyglucosides, alkyl sulfates, alkyl sulfonates, alkyl ester sulfonates, alkyl aromatic sulfonates, alkyl betaine, alkyl amido betaines, or combinations thereof), hydrocarbon based dispersants (e.g., lignosulfonates, iminodisuccinates, polyaspartates, or combinations thereof), amino acids, proteins, or combinations thereof.

Examples of biocides include, but are not limited to, oxidizing and non-oxidizing biocides. Examples of non-oxidizing biocides include, for example, aldehydes (e.g., formaldehyde, glutaraldehyde, acrolein, or combinations thereof), amine-type compounds (e.g., quaternary amine compounds, cocodiamine, or a combination thereof), halogenated compounds (e.g., bronopol, 2-2-dibromo-3-nitrilopropionamide (DBNPA), or a combination thereof), sulfur compounds (e.g., isothiazolone, carbamates, metronidazole, or a combination thereof), quaternary phosphonium salts (e.g., tetrakis(hydroxymethyl)phosphonium sulfate (THPS)), or combinations thereof.

Examples of oxidizing biocides include sodium hypochlorite, trichloroisocyanuric acids, dichloroisocyanuric acid, calcium hypochlorite, lithium hypochlorite, chlorinated hydantoins, stabilized sodium hypobromite, activated sodium bromide, brominated hydantoins, chlorine dioxide, ozone, peroxides, or combinations thereof.

The methods and apparatus disclosed herein measure the polarization resistance of a field fluid or a sample of the field fluid from a field system to determine the concentration of the chemical (e.g., a corrosion inhibitor, a scale inhibitor, an asphaltene inhibitor, a gas hydrate inhibitor, or a biocide) in the field fluid.

Embodiments of the methods include a calibration stage, a field-testing stage, or both. Generally the calibration stage takes place before the field-testing stage so that calibration equations are present when field fluids are tested for a chemical so as to convert a polarization resistance of the field fluid into a concentration for the chemical in the field fluid. The calibration stage of the method can be performed independently of the field-testing stage of the method.

### Calibration Stage:

In the calibration stage, the methods can include adding a chemical to each of a plurality of base fluids at a test reference point in time to form a plurality of calibration fluids. In aspects, the base fluid is similar to or the same as the field fluid. For example, if the field fluid is a composition in a gas phase, then the base fluid is a similar or same composition in the gas phase. If the field fluid is a composition in a liquid phase, then the base fluid is a similar or same composition in the liquid phase.

In some aspects, a single volume of base fluid composition can be obtained. The polarization resistance of the single volume of base fluid can be determined without presence of the chemical (e.g., a blank calibration fluid). Then, the chemical can be added to the fluid in known and different stair-stepped amounts that are each tested, with measurements taken on each stair-stepped amount of chemical in the fluid. For example, the chemical can be in a first amount and the fluid having the chemical in a first concentration can be tested for polarization resistance with the first concentration of chemical present. Then, more of the chemical can be added to the fluid having the first concentration of chemical to raise the amount of chemical to a second concentration, and the fluid can be tested for polarization resistance with the second concentration of chemical present; and so on.

In alternative aspects, multiple calibration fluids are formed so that different known concentrations of the chemical present in the fluids may be tested for polarization resistance. Multiple portions of the same base fluid can be obtained, with each having a different concentration of the chemical added thereto to form a calibration fluid (e.g., 0, 10, 20, 30, 40, 50, 60, 70, 80, 90, and 100 μm; or other appropriate variety and range of concentrations depending on the chemical). Each calibration fluid then can be tested for polarization resistance versus time.

The test reference point in time can be when the chemical additive is added to the calibration fluid.

In the calibration stage, the methods can include generating a polarization resistance curve versus time for each of the calibration fluids. Each polarization resistance curve for each of the calibration fluids starts at a common start time relative to the test reference point in time. For example, for the calibration fluids made by successively stair-stepping the concentration of the chemical in the fluid, the common start time can be when the chemical is added to the fluid in the respective additional amount. Alternatively, the common start time can be a start time before the chemical is added, such as 5 minutes prior to stair-stepped addition, so long as all start times for the polarization resistance curves start 5 minutes before the additional amount of the chemical is added to the fluid. In the example of multiple calibration fluids having different chemical concentrations added to the fluid, the laboratory personnel can add the chemical to the fluid in the respective known concentration at an appropriate time that the laboratory equipment is ready to begin recording polarization resistance values at the common start time (before or at time of chemical addition to each base fluid).

A polarization resistance curve is generated for each of the calibration fluids, and in some cases, also for a blank of the base fluid having no chemical, also referred to as a calibration fluid.

Polarization resistance curves are generated using an apparatus described herein. The apparatus can be a laboratory environment apparatus, and include a vessel, an electrochemical probe coupled to the vessel, a potentiostat (or other equipment suitable for the measurements) operably connected to the electrochemical probe, and a computer operably connected to the potentiostat.

The vessel can be any vessel, such as a beaker, flask, vial, test tube, bubble test cell, cup, and the like. The potentiostat can be any device that measures potential difference between electrodes in the electrochemical probe. The computer has one or more processors and a memory having instructions stored thereon that cause the one or more processors to perform any combination of the steps of the methods described herein, including generating the polarization resistance curves. In some embodiments, the electrochemical probe has a working electrode, a reference electrode, and a counter electrode. In aspects, the working electrode can be formed of a carbon steel or stainless steel (optionally covered with a layer of conducting polymer), the reference electrode can be formed of stainless steel, carbon steel, or Hastelloy (optionally covered with a layer of conducting polymer), and the counter electrode can be formed of stainless steel, carbon steel, or Hastelloy (optionally covered with a layer of conducting polymer). In other embodiments, the electrochemical probe can be embodied as array of electrochemical sensors, such as an electronic nose (e-nose, for gas phase fluids) or electronic tongue (e-tongue, for liquid phase fluids). Each of the sensors can include one or more electrodes for measurements made by linear polarization resistance (e.g., potentiometric measurements), measurements made via electrochemical impedance spectroscopy, measurements made by chronoamperometry, or a combination thereof.

An example of how polarization resistance curves are generated includes polarizing a working electrode of the electrochemical probe (or at least one sensor of an array described above) with a potential sweep from a negative voltage value to a positive voltage value relative to the open circuit potential (also known as OCP) of the electrochemical probe. The potential of the working electrode can be referenced to the reference electrode and induced via the potentiostat. The induced potential and current can be measured and recorded by the computer at preset time intervals, such as every 1, 2, 3, 4, 5, 6, 7, 8, 9, 10, 15, 20, 25, or 30 seconds. The polarization resistance can be calculated from the induced potential and the current measurements at each preset time interval of measurement. The series of polarization resistance values versus time form the polarization resistance curve for the respective fluid which is tested. The curve starts at the common start time relative to the test reference point in time and lasts as long as necessary for the particular application. For example, for corrosion inhibitors in oil and gas fluids, the polarization resistance curves for the calibration fluids can be in a range of from 1 hour to 24 hours; alternatively, from 1 day to 30 days. Other chemical additives may have other suitable time ranges for their respective application, such as on order of seconds, minutes, hours, or days.

In the calibration stage, the methods can include generating a calibration equation based on a calibration polarization resistance value in each of the polarization resistance curves at a calibration point in time that occurs after the test reference point in time. Any number of calibration equations can be generated. An example of a calibration equation for a calibration point in time of 1 hour is now described for polarization resistance curves generated for calibration fluids having 0 ppm, 10 ppm, 20 ppm, and 30 ppm of a chemical additive over a time period of 3 hours, where the 3 hour time period for each curve begins at a common start time relative to the test reference point in time. The table below describes the test timing to generate the curves:

| | |
|---|---|
| Calibration fluid #1 | Day 1: Electrochemical probe placed into a base fluid, which is a |
| containing 0 ppm of | blank calibration fluid because there is no chemical (0 ppm) |
| chemical | present. At 10:30 am on Day 1 potential is applied, and |
| | measurements are taken every 15 minutes for 4 hours. |
| | Polarization resistance values are calculated for each |
| | measurement taken over the 4 hours. The series of values |
| | versus time is the polarization resistance curve for the calibration |
| | fluid #1. Because of calibration fluids #2-4 have a time of 1 hour |
| | (as described below) before chemical is added to the base fluid, |
| | 11:30 am on Day 1 is the test reference point in time for |
| | calibration fluid #1. The 3-hour time period for the polarization |
| | resistance curve would include values calculated from data |
| | measured from 11:30 am to 2:30 pm on Day 1. To generate a |
| | calibration equation for a calibration point in time of 1 hour |
| | relative to the reference point in time (i.e., a calibration time |
| | period of 1 hour), the polarization resistance value at 12:30 pm |
| | on Day 1 would be selected from the polarization resistance |
| | curve of calibration fluid #1. |
| Calibration fluid #2 | Day 2: Base fluid is placed in a vessel. Electrochemical probe |
| containing 10 ppm | placed into the base fluid at 9:30 am and potential applied to take |
| of chemical | measurements in the base fluid. At 10:30 am, chemical is added |
| | to the base fluid to a concentration of 10 ppm of chemical, |
| | forming the 10 ppm calibration fluid. 10:30 am on Day 2 is the test |
| | reference point in time for calibration fluid #2. Measurements |
| | taken every 15 minutes for 3 hours after the test reference point |
| | in time. Polarization resistance values are calculated for each |
| | measurement taken over the 3 hours. The series of values |
| | versus time is the polarization resistance curve for the calibration |
| | fluid #2. To generate a calibration equation for a calibration point |
| | in time of 1 hour relative to the reference point in time (i.e., a |
| | calibration time period of 1 hour), the polarization resistance |
| | value at 11:30 am on Day 2 would be selected from the |
| | polarization resistance curve of calibration fluid #2. |
| Calibration fluid #3 | Day 3: Base fluid is placed in a vessel. Electrochemical probe |
| containing 20 ppm | placed into the base fluid at 9:30am and potential applied to take |
| of chemical | measurements in the fluid. At 10:30 am, chemical is added to the |
| | base fluid to a concentration of 20 ppm of chemical, forming the |
| | 20 ppm calibration fluid. 10:30am on Day 3 is the test reference |
| | point in time for calibration fluid #3. Measurements taken every |
| | 15 minutes for 3 hours after the test reference point in time. |
| | Polarization resistance values are calculated for each |
| | measurement taken over the 3 hours. The series of values |
| | versus time is the polarization resistance curve for the calibration |
| | fluid #3. To generate a calibration equation for a calibration point |
| | in time of 1 hour relative to the reference point in time (i.e., a |
| | calibration time period of 1 hour), the polarization resistance |
| | value at 11:30 am on Day 3 would be selected from the |
| | polarization resistance curve of calibration fluid #3. |
| Calibration fluid #4 | Day 4: Base fluid is placed in a vessel. Electrochemical probe |
| containing 20 ppm | placed into the base fluid at 9:30 am and potential applied to take |
| of chemical | measurements in the fluid. At 10:30 am, chemical is added to the |
| | base fluid to a concentration of 30 ppm of chemical, forming the |
| | 30 ppm calibration fluid. 10:30 am on Day 4 is the test reference |
| | point in time for calibration fluid #4. Measurements taken every |
| | 15 minutes for 3 hours after the test reference point in time. |
| | Polarization resistance values are calculated for each |
| | measurement taken over the 3 hours. The series of values |
| | versus time is the polarization resistance curve for the calibration |
| | fluid #4. To generate a calibration equation for a calibration point |
| | in time of 1 hour relative to the reference point in time (i.e., a |
| | calibration time period of 1 hour), the polarization resistance |
| | value at 11:30 am on Day 4 would be selected from the |
| | polarization resistance curve of calibration fluid #4. |

Continuing this example, the value of the polarization resistance at 1 hour calibration point in time on each curve for each fluid (11:30 am on Day 1 for calibration fluid #1, 11:30 am on Day 2 for calibration fluid #2, 11:30 am on Day 3 for calibration fluid #3, and 11:30 am on Day 4 for calibration fluid #4) is selected. A total of four concentration/polarization resistance data point pairs are selected in this example: 1) the polarization resistance value of calibration fluid #1 at 1 hour calibration point in time (11:30 am on Day 1) and the concentration of 0 ppm of the chemical, 2) the polarization resistance value of calibration fluid #2 at 1 hour calibration point in time (11:30 am on Day 2) and the concentration of 10 ppm of the chemical, 3) the polarization resistance value of calibration fluid #3 at 1 hour calibration point in time (11:30 am on Day 3) and the concentration of 20 ppm of the chemical, and 4) the polarization resistance value of calibration fluid #4 at 1 hour calibration point in time (11:30 am on Day 4) and the concentration of 30 ppm. The computer of the apparatus then generates the calibration equation by which a polarization resistance of a field sample can be input to calculate a concentration of the chemical in a field sample (or any sample of the same or similar base fluid). For example, the computer can assign the concentrations as Y values and the polarization resistance values as X values for an X-Y graph, and then fit the four data points to a regression equation, the regression equation being the calibration equation described herein. Alternatively, the calibration equation can be generated manually by a person using another computer, such as using a spreadsheet software, where a X-Y graph is generated from the concentration/polarization resistance data point pairs, and a regression equation is fit to the data points—the regression equation being the calibration equation. Generally, the calibration equation calculates a concentration of the chemical in sample of a field fluid when a sample polarization resistance value that was determined for the sample of the field fluid is input to the calibration equation.

The common start time of the above example is: 1) 10:30 am on Day 1 for calibration fluid #1, 2) 9:30 am on Day 2 for calibration fluid #2, 3) 9:30 am on Day 3 for calibration fluid #3, and 4) 9:30 am on Day 4 for calibration fluid #4. In alternative embodiments, the potential could have been applied to the calibration fluids #2-4 at the time of chemical addition, making the common start time for calibration fluids #2-4 the same time as the reference point in time.

While polarization resistance values can be calculated based on potentiometric measurements as described above, other types of measurements can be utilized to calculate the polarization resistance values. Examples of measurements that can be taken are measurements made by linear polarization resistance (e.g., potentiometric measurements), measurements made via electrochemical impedance spectroscopy, measurements made by chronoamperometry, or a combination thereof.

### Field-Testing Stage:

The field-testing stage of the methods disclosed herein can include determining a field reference point in time at which a chemical is introduced into a field fluid. In aspects, the field reference point in time is the time when the chemical is introduced into the field fluid. Determining the field reference point in time is important so that the time period between the time when chemical is introduced to the field fluid in the field system (the field reference point in time) and the time when the polarization resistance value of the field sample is determined (the measurement point in time) matches (is equal to) the time period for one of the calibration equation(s) that is generated in the calibration stage. The field reference point in time can be obtained from equipment (such as a control computer) that saves information about when the chemical is introduced into the field system, and in some cases, the amount of the chemical that is introduced. Control equipment by which chemicals are introduced into field systems are known in the art, as is sampling equipment by which samples of field fluids are withdrawn from a flow line or vessel (for example) in the field system.

By example only, an oil and gas pipeline may have a corrosion inhibitor dosing pump or valve at a location on the pipeline. The pump can be turned on, or the valve opened, to allow an amount of the corrosion inhibitor to be introduced into the field fluid in the pipeline at the dosing location. The corrosion inhibitor then flows with the field fluid in the pipeline. At a location on the pipeline that is downstream of the dosing location, a sample can be withdrawn from the sampling equipment. The sample can be obtained from the field system in which the field fluid is contained according to any technique for obtaining samples from a field system. For example, in a water treatment system, a sample of the water in a vessel or piping in the system can be obtained through sample line and valve connected to the system. The valve can be opened to allow a small volume of the field fluid to flow into a sample collection container, such as a beaker, flask, vial, test tube, bubble test cell, cup, and the like.

The field-testing stage of the methods disclosed herein can include determining a sample polarization resistance value in a sample of the field fluid at a measurement point in time. An apparatus similar to that described above for the calibration stage, or the same apparatus in the laboratory environment, can be used to place an electrochemical probe in the field sample. Generally, the apparatus can include a vessel, an electrochemical probe coupled to the vessel, a potentiostat (or other equipment suitable for the measurements) operably connected to the electrochemical probe, and a computer operably connected to the potentiostat.

An example of how the sample polarization resistance value can be determined includes placing the electrochemical probe into the sample of the field fluid (e.g., the water phase of a field fluid) and polarizing a working electrode of the electrochemical probe (or at least one sensor of an array described above) with a potential sweep from a negative voltage value to a positive voltage value relative to the open circuit potential (also known as OCP) of the electrochemical probe. The potential of the working electrode can be referenced to the reference electrode and induced via the potentiostat. The induced potential and current can be measured and recorded by the computer of the apparatus. The sample polarization resistance value can be calculated from the induced potential and the current measurement.

The time that the measurements are taken (upon applying the potential on the probe) is the measurement point in time, and the sample polarization resistance value can be associated with the measurement point in time.

Thus, at this point in the field-testing stage, the field reference point in time and the measurement point in time have been determined.

The field-testing stage of the methods disclosed herein can also include inputting the sample polarization resistance value into one of the calibration equations generated in the calibration stage of the methods. One of the calibration equations, or perhaps the only calibration equation for a chemical, is selected for inputting the sample polarization resistance value into the calibration equation. The calibration equation is selected according to the time period (or elapsed time) between the test reference point in time and the calibration point in time for the data points used to generate the calibration equation. That is, the calibration equation having a time period (or calibration elapsed time) that is equal to the time period (or field elapsed time) between the field reference point in time and the measurement point in time, is selected. Matching the field elapsed time and the calibration elapsed time is important so that the calibration equation can calculate a correct value for the concentration of the chemical in sample of the field fluid (which is indicative of the concentration of the chemical in the field fluid in the field system).

The sample polarization resistance value is input into the calibration equation. Inputting can be performed by the computer or manually by a human that receives the sample polarization resistance value and inputs that value into the calibration equation on a calculator on running on another computer (such as in a spreadsheet software).

The field-testing stage of the methods disclosed herein can include determining, based on the calibration equation, a field concentration of the chemical in the sample of the field fluid. The computer that calculates the concentration based on the calibration equation determines the field concentration of the chemical in the sample of the field fluid. The methods utilizing the apparatus thus measure a single polarization resistance value in the sample from field fluid of the field system. The measurement is taken for a time period relative to a reference time that is the same time period for data points having concentration and polarization resistance value pairs that are used to generate the calibration equation in the calibration stage described herein. Using the calibration equation(s) eliminates the need to perform a time-intensive experiment to generate a polarization resistance curve for the field sample, since only a single value for polarization resistance is determined. Because no polarization resistance curve is generated for the field sample, the need to compare the curve to standard curves is avoided, and thus, the concentration of the chemical in the field system is determined faster than techniques which compare field sample curves to standard curves.

After determining the field concentration of the chemical in the sample of the field fluid, methods disclosed herein can include introducing or not introducing, based on the field concentration, an additional amount of the chemical into the field system (e.g., the oil and gas system, the geothermal system, the carbon capture system, the carbon storage system, the hydrogen transport system, the water transport system, or the water treatment system from which the sample is obtained). When additional amount of the chemical is needed based on the field concentration determined, such as when the field concentration is below a threshold concentration for the field system, the chemical can be introduced into the field system via the dosing equipment described above. Alternatively, if the amount of the chemical determined in this step is above the threshold concentration, then the dosing system can refrain from introducing additional chemical into the field system. Because the polarization resistance value is determined faster according to the disclosed methods and apparatus, the action of introducing or not introducing the additional amount of chemical can be taken faster than techniques which generate an entire polarization resistance curve for the field sample.

### EXAMPLES

The following examples demonstrate an embodiment of the techniques disclosed herein by which polarization resistance curves of calibration fluids are used to create a calibration equation which is then used to determine the concentration of a chemical in a field fluid.

Seven calibration fluids were prepared, all liquids, referred to as Calibration Liquids 1 to 7. Each calibration liquid contained a base fluid that was a brine of 3 wt % sodium chloride in water. The chemical for which concentration was calibrated and determined was a corrosion inhibitor. The corrosion inhibitor had 25 wt % quaternary ammonium compounds, 6 wt % first organic sulfur compound, 4 wt % phosphoric acid esters, 6 wt % second organic sulfur compound, 2.5 wt % alkylamine/polyamine, and 56.5 wt % solvents. This composition for the corrosion inhibitor was exemplary, and other compositions of corrosion inhibitor could have been used. Moreover, while a corrosion inhibitor was used to demonstrate these examples, a scale inhibitor, an asphaltene inhibitor, a gas hydrate inhibitor, or a biocide could have been used as the chemical.

Calibration Liquid 1 was a blank and contained only the base fluid (the brine). Calibration Liquids 2 to 7 contained the base fluid (the brine) and an amount of the corrosion inhibitor indicated below:

| | Concentration |
|---|---|
| Calibration | of Corrosion |
| Liquid | Inhibitor |
| (#) | (ppm) |
| 1 | 0 |
| 2 | 5 |
| 3 | 10 |
| 4 | 30 |
| 5 | 30 |
| 6 | 50 |
| 7 | 70 |

Experiments to generate polarization resistance curves for each of the Calibration Liquids 1 to 7 were performed with an apparatus having a standard bubble test cell, an electrochemical probe, a potentiostat connected to the probe, and a computer connected to the potentiostat. The electrochemical probe had a working electrode (C1018 carbon steel), a reference electrode (Hastelloy), and a counter electrode (Hastelloy). The following procedure was followed for each experiment:

### Procedure:

The standard bubble test cell was filled with the based fluid (a brine deaerated with carbon dioxide), and the base fluid was heated to 80 °C while at atmospheric pressure. The electrochemical probe was added to the bubble test cell and connected to a potentiostat.

The brine in each experiment was allowed to sit for 3 hours in the bubble test cell. At 3 hours, the corrosion inhibitor was added in the respective concentration for Calibration Liquids 2 to 7 (and not added for Calibration Liquid 1 which was used as a blank or benchmark experiment).

Polarization resistance measurements were made approximately every 15 minutes. Measurements were made by polarizing the working electrode with a potential swept from −10 mV versus its open circuit potential (OCP) to +10 mV OCP at a scan rate of 40 mV/minute. The potential of the working electrode was referenced to the reference electrode and induced via the potentiostat. The resulting current measured between the working electrode and the counter electrode via the potentiostat was recorded and logged on the data analysis software. The polarization resistance was calculated from the induced potential and the current measurements.

### Results:

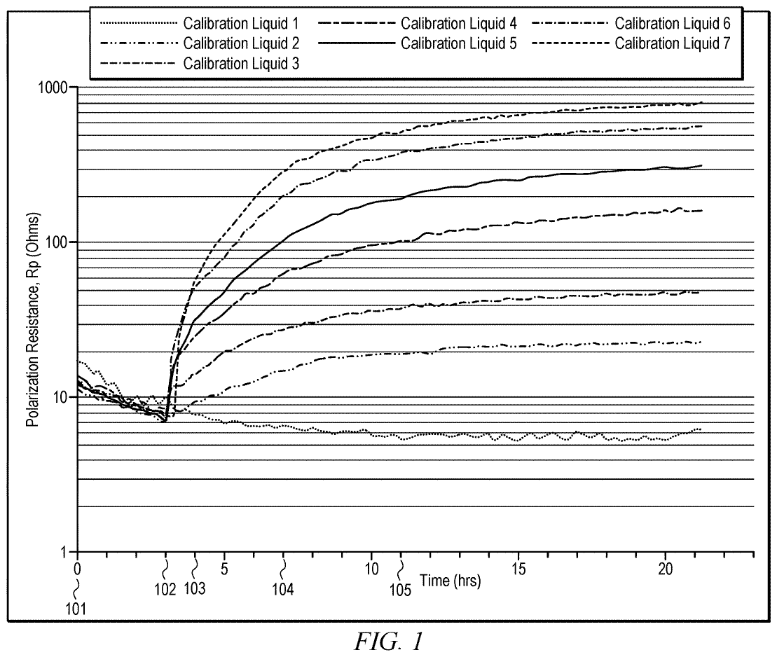

FIG. 1 illustrates a graph of polarization resistance curves for the Calibration Liquids 1 to 7. The time (labeled 101) of 0 hour in the graph of FIG. 1 is the common start time for Calibration Liquids 1 to 7. The time (labeled 102) of 3 hours on the X-axis in the graph of FIG. 1 is the test reference point in time, and this time 102 is when the corrosion inhibitor was added to the base fluid to form each of the Calibration Liquids 2 to 7. The calibration time period between calibration point in time 103 of hour 4 and the test reference point in time 102 at hour 3 on the X-axis in the graph of FIG. 1 is 1 hour. The calibration time period between calibration point in time 104 of hour 7 and the test reference point in time 102 at hour 3 on the X-axis in the graph of FIG. 1 is 4 hours. The calibration time period between calibration point in time 105 of hour 11 and the test reference point in time 102 at hour 3 on the X-axis in the graph of FIG. 1 is 8 hours. Hours 4, 7, and 11 were chosen to make calibration equations. Other hours of calibration point int time in the graph could have been chosen for the calibration time periods.

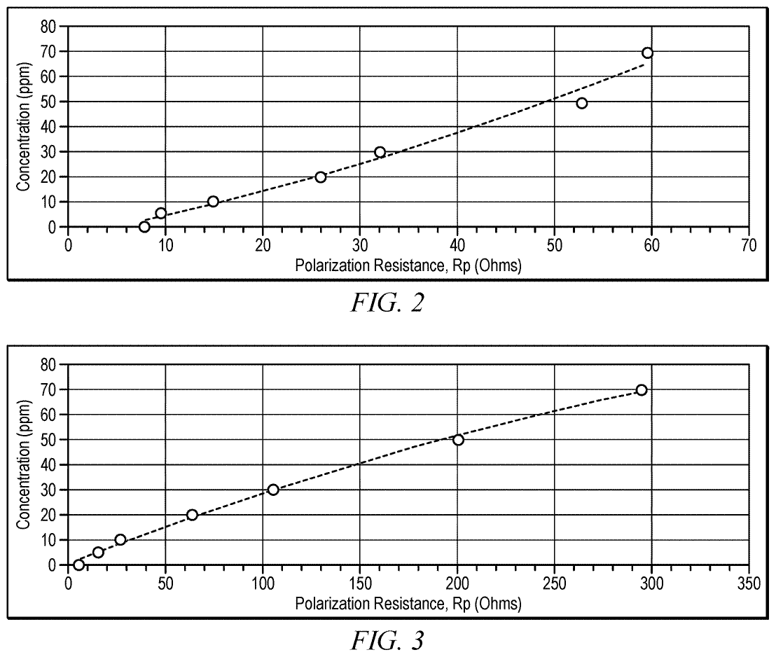

FIG. 2 illustrates a graph of chemical concentration versus polarization resistance, where the polarization resistance values are taken from the curves in FIG. 1 at a time of 4 hours in FIG. 1. The 1-hour calibration equation for the data points in FIG. 2 is:

C_{Chemical}=0.007Rp²+0.7625Rp−4.0962(R²=0.9846).

FIG. 3 illustrates a graph of chemical concentration versus polarization resistance, where the polarization resistance values are taken from the curves in FIG. 1 at a time of 7 hours in FIG. 1. The 4-hour calibration equation for the data points in FIG. 2 is:

C_{Chemical}=−0.0002Rp²+0.3007Rp+0.3318(R²=0.9969).

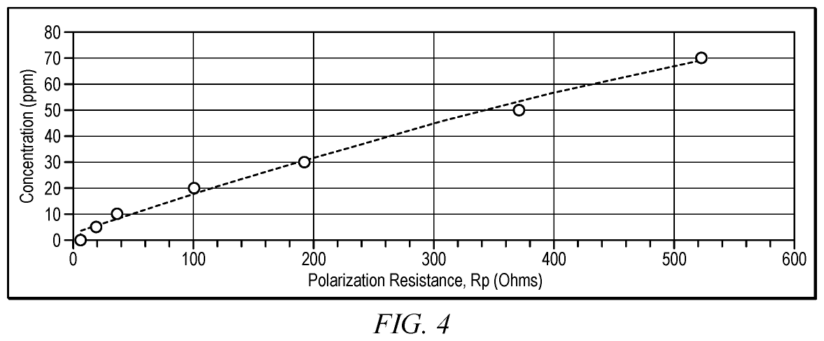

FIG. 4 illustrates a graph of chemical concentration versus polarization resistance, where the polarization resistance values are taken from the curves in FIG. 1 at a time of 11 hours in FIG. 1. The 8-hour calibration equation for the data points in FIG. 2 is:

C_{Chemical}=(−5E−05)Rp²+0.1539Rp−2.3297(R²=0.9926).

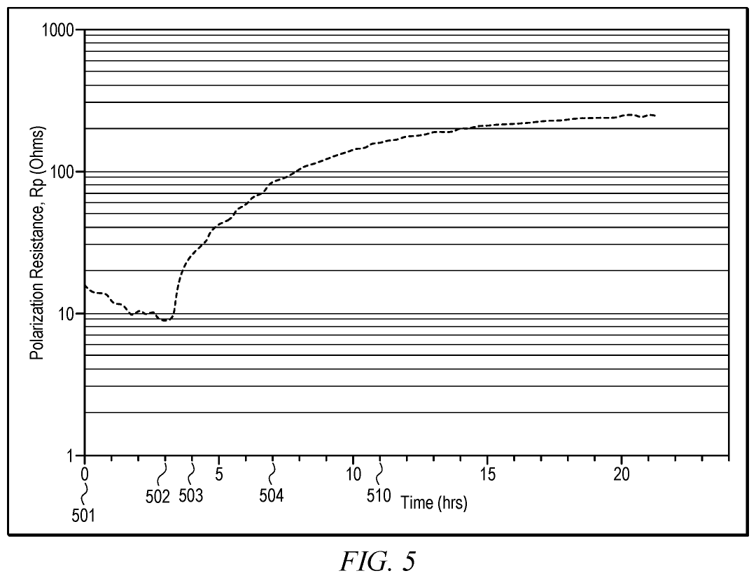

FIG. 5 illustrates a graph of a polarization resistance curve for the test liquid in the examples. The polarization resistance curve was generated to validate or invalidate the calibration equations determined for FIGS. 2, 3, and 4. The same procedure for the Calibration Fluids was followed for the test liquid. A test liquid containing the base fluid and 25 ppm of the corrosion inhibitor was tested. The bubble test cell was filled with base fluid, the electrochemical probe placed in the base fluid, measurements taken starting at time=0 hours (labeled 501) in the graph of FIG. 5. The base fluid was allowed to sit for 3 hours (labeled 502). At 3 hours, 25 ppm of the corrosion inhibitor was added. Measurements with the apparatus continued for the next 17 hours (until total time elapsed of about 20 hours).

Even though it was known that the test liquid contained 25 ppm of the corrosion inhibitor for purposes of validation, no polarization resistance values in the polarization resistance curve in FIG. 5 were used to generate a calibration equation. Instead, the times labeled at 503, 504, and 505 were used to find the polarization resistance values of the test liquid for validation. Times labeled at 503, 504, and 505 corresponded to the times 103, 104 and 105 in FIG. 1, and the data points in FIG. 1 at times 103, 104, and 105 were the data points in FIGS. 2, 3, and 4 from which the calibration equations were generated, so that the field time period for the simulated polarization resistance values were equal to the calibration time periods of the calibration equations. For field samples, one sample polarization resistance value can be determined and input to one calibration equations. These examples used three calibration equations for validation purposes.

The time period between point in time 503 of hour 4 (a simulated measurement point in time for validation) and the point in time 502 at hour 3 (a simulated field reference point in time for validation) on the X-axis in the graph of FIG. 5 corresponds to the time period between calibration point in time 103 of hour 4 and the test reference point in time 102 at hour 3 on the X-axis in the graph of FIG. 1, which is 1 hour. Thus, the 1-hour calibration equation generated from the data points in FIG. 2 was used to calculate the concentration of the corrosion inhibitor in the test liquid using the polarization resistance value of the test liquid at time 503 of hour 4. The polarization resistance value of FIG. 5 at time 503 (hour 4) was 29.221 Ohms. Inputting 29.221 Ohms into the 1-hour calibration equation above, the calculated concentration of corrosion inhibitor was 24.16 ppm, a 3.4% error from 25 ppm.

The time period between point in time 504 of hour 7 (another simulated measurement point in time for validation) and the point in time 502 at hour 3 (the simulated field reference point in time for validation) on the X-axis in the graph of FIG. 5 corresponds to the time period between calibration point in time 104 of hour 7 and the test reference point in time 102 at hour 3 on the X-axis in the graph of FIG. 1, which is 4 hours. Thus, the 4-hour calibration equation generated from the data points in FIG. 3 was used to calculate the concentration of the corrosion inhibitor in the test liquid using the polarization resistance value of the test liquid at time 504 of hour 7. The polarization resistance value of FIG. 5 at time 504 (hour 7) was 84.047 Ohms. Inputting 84.047 Ohms into the 4-hour calibration equation above, the calculated concentration of corrosion inhibitor was 24.19 ppm, a 3.2% error from 25 ppm.

The time period between point in time 505 of hour 11 (another simulated measurement point in time for validation) and the point in time 502 at hour 3 (the simulated field reference point in time for validation) on the X-axis in the graph of FIG. 5 corresponds to the time period between calibration point in time 105 of hour 11 and the test reference point in time 102 at hour 3 on the X-axis in the graph of FIG. 1, which is 8 hours. Thus, the 8-hour calibration equation generated from the data points in FIG. 4 was used to calculate the concentration of the corrosion inhibitor in the test liquid using the polarization resistance value of the test liquid at time 505 of hour 11. The polarization resistance value of FIG. 5 at time 505 (hour 11) was 160.762 Ohms. Inputting 160.762 Ohms into the 8-hour calibration equation above, the calculated concentration of corrosion inhibitor was 25.78 ppm, a 3.1% error from 25 ppm.

These examples demonstrate that calibration equations can be utilized to quickly determine concentration of chemical in a sample of field fluid faster than other techniques, while having only about 3% error when seven calibration fluids are used to determine the calibration equation. Using more calibration fluids to determine calibration equations can reduce the error.

While these examples used a brine as a base fluid, which simulates a water phase of liquid from a field system, the calibration testing can also be used on field fluids that have water phase and hydrocarbon phase. Samples of such mixtures typically partition into water phase and hydrocarbon phase. Base fluids having both the hydrocarbon phase and water phase can be used. The chemical can be added to the base fluids. However, the personnel testing the fluid can first allow the water phase and hydrocarbon phase to partition, and then use the water phase containing the chemical as the calibration fluid. The polarization resistance curves and calibration equations can then be made form the water phases as the calibration fluids. When testing a field sample, the field sample can be allowed to partition, and the water phase used as the testing sample to measure potential and calculate the polarization resistance of the field fluid, which when the relevant reference time and calibration times are determined, the polarization resistance of the field fluid can be entered into a corresponding calibration equation to calculate the concentration of the chemical in water phase of the field fluid.

### ADDITIONAL DESCRIPTION

Aspect 1. A method comprising: adding a chemical to each of a plurality of calibration fluids at a test reference point in time for each of the plurality of calibration fluids, wherein a concentration of the chemical in each of the plurality of calibration fluids is known and is different in each of the plurality of calibration fluids; generating a polarization resistance curve versus time for each of the plurality of calibration fluids, wherein the polarization resistance curve for each of the plurality of calibration fluids starts at a common start time relative to the test reference point in time; and generating a calibration equation based on a calibration polarization resistance value in each of the polarization resistance curves at a calibration point in time that occurs after the test reference point in time; wherein the calibration equation calculates a concentration of the chemical in a sample of a field fluid when a sample polarization resistance value that was determined for the sample of the field fluid is input to the calibration equation.
Aspect 2. The method of Aspect 1, further comprising: determining a field reference point in time at which the chemical was introduced into a field fluid; determining a sample polarization resistance value in a sample of the field fluid at a measurement point in time; inputting the sample polarization resistance value into the calibration equation; and determining, based on the calibration equation, a field concentration of the chemical in the sample of the field fluid.
Aspect 3. The method of Aspect 2, wherein a first time period between the field reference point in time and the measurement point in time is equal to a second time period between the test reference point in time and the calibration point in time.
Aspect 4. The method of Aspect 2, wherein the sample of the field fluid is obtained from an oil and gas system, a geothermal system, a carbon capture system, a carbon storage system, a hydrogen transport system, a water transport system, or a water treatment system.
Aspect 5. The method of Aspect 4, further comprising: introducing or not introducing, based on the field concentration, an additional amount of the chemical into the oil and gas system, the geothermal system, the carbon capture system, the carbon storage system, the hydrogen transport system, the water transport system, or the water treatment system.
Aspect 6. The method of Aspect 4, wherein the sample is obtained from a pipeline, a process flow line, a valve, a separation vessel, a storage vessel, or a combination thereof.
Aspect 7. The method of Aspect 1, wherein the chemical is a corrosion inhibitor, a scale inhibitor, a gas hydrate inhibitor, or a biocide.
Aspect 8. The method of Aspect 1, wherein the calibration polarization resistance values and the sample polarization resistance value are generated using a linear polarization resistance technique, electrochemical impedance spectroscopy, or chronoamperometry.
Aspect 9. The method of Aspect 1, wherein each of the plurality of calibration fluids comprises a brine, wherein the calibration polarization resistance values are generated from the brine.
Aspect 10. The method of Aspect 1, wherein the sample of the field fluid comprises a brine and a hydrocarbon, wherein the sample polarization resistance value is generated from the brine.
Aspect 11. The method of Aspect 1, wherein the polarization resistance curve is generated for each of the plurality of calibration fluids using an electrochemical probe placed into a water phase of each of the plurality of calibration fluids.
Aspect 12. The method of Aspect 11, wherein the electrochemical probe comprises an array of electrochemical sensors.
Aspect 13. The method of Aspect 11, wherein the electrochemical probe comprises a working electrode, a reference electrode, and a counter electrode.
Aspect 14. The method of Aspect 13, wherein the working electrode comprises carbon steel or stainless steel (each type optionally covered with a layer of conducting polymer), the reference electrode comprises stainless steel, carbon steel, or Hastelloy (each type optionally covered with a layer of conducting polymer), and the counter electrode comprises stainless steel, carbon steel, or Hastelloy (each type optionally covered with a layer of conducting polymer).
Aspect 15. An apparatus comprising: a test vessel; an electrochemical probe coupled to the test vessel; a potentiostat; and a computer operably connected to the electrochemical probe, wherein the computer has one or more processors and a memory having instructions stored thereon that cause the one or more processors to: generate a polarization resistance curve versus time for each of a plurality of calibration fluids, wherein the polarization resistance curve for each of the plurality of calibration fluids starts at a common start time relative to a test reference point in time; and generate a calibration equation based on a calibration polarization resistance value in each of the polarization resistance curves at a calibration point in time that occurs after the test reference point in time; wherein the calibration equation calculates a concentration of a chemical in a sample of a field fluid when a sample polarization resistance value that was determined for the sample of the field fluid is input to the calibration equation.
Aspect 16. The apparatus of Aspect 15, wherein the chemical is a corrosion inhibitor, a scale inhibitor, a gas hydrate inhibitor, or a biocide.
Aspect 17. The apparatus of Aspect 15, wherein the calibration polarization resistance values and the sample polarization resistance value are generated using a linear polarization resistance technique, electrochemical impedance spectroscopy, or chronoamperometry.
Aspect 18. The apparatus of Aspect 15, wherein each of the plurality of calibration fluids comprises a brine, wherein the calibration polarization resistance values are generated from the brine.
Aspect 19. The apparatus of Aspect 15, wherein the sample of the field fluid comprises a brine and a hydrocarbon, wherein the sample polarization resistance value is generated from the brine.
Aspect 20. The apparatus of Aspect 15, wherein the polarization resistance curve is generated for each of the plurality of calibration fluids using an electrochemical probe placed into a water phase of each of the plurality of calibration fluids.
Aspect 21. The apparatus of Aspect 20, wherein the electrochemical probe comprises an array of electrochemical sensors.
Aspect 22. The apparatus of Aspect 20, wherein the electrochemical probe comprises a working electrode, a reference electrode, and a counter electrode.
Aspect 23. The apparatus of Aspect 22, wherein the working electrode comprises carbon steel or stainless steel (each type optionally covered with a layer of conducting polymer), the reference electrode comprises stainless steel, carbon steel, or Hastelloy (each type optionally covered with a layer of conducting polymer), and the counter electrode comprises stainless steel, carbon steel, or Hastelloy (each type optionally covered with a layer of conducting polymer).
Aspect 24. A method comprising: determining a reference point in time at which a chemical was introduced into a field fluid; determining a sample polarization resistance value in a sample of the field fluid at a measurement point in time; inputting the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and determining, based on the calibration equation, the concentration of the chemical in the sample of the field fluid.
Aspect 25. The method of Aspect 24, further comprising: introducing or not introducing, based on the field concentration, an additional amount of the chemical into an oil and gas system, a geothermal system, a carbon capture system, a carbon storage system, a hydrogen transport system, a water transport system, or a water treatment system.
Aspect 26. The method of Aspect 24, wherein the calibration time is a calibration time period between a test reference point in time and a calibration point in time for concentration/polarization resistance data point pairs from which the calibration equation is generated.
Aspect 27. The method of Aspect 24, wherein the sample of the field fluid is obtained from an oil and gas system, a geothermal system, a carbon capture system, a carbon storage system, a hydrogen transport system, a water transport system, or a water treatment system.
Aspect 28. The method of Aspect 24, wherein the sample is obtained from a pipeline, a process flow line, a valve, a separation vessel, a storage vessel, or a combination thereof.
Aspect 29. The method of Aspect 24, wherein the chemical is a corrosion inhibitor, a scale inhibitor, a gas hydrate inhibitor, or a biocide.
Aspect 30. The method of Aspect 24, wherein the sample polarization resistance value is generated using a linear polarization resistance technique, electrochemical impedance spectroscopy, or chronoamperometry.
Aspect 31. The method of Aspect 24, wherein the field fluid comprises a brine.
Aspect 32. The method of Aspect 24, wherein the field fluid comprises a brine and a hydrocarbon, wherein the sample polarization resistance value is determined from the brine.
Aspect 33. The method of Aspect 24, wherein the sample polarization resistance value is determined using an electrochemical probe placed into the sample of the field fluid.
Aspect 34. The method of Aspect 33, wherein the electrochemical probe comprises an array of electrochemical sensors.
Aspect 35. The method of Aspect 33, wherein the electrochemical probe comprises a working electrode, a reference electrode, and a counter electrode.
Aspect 36. The method of Aspect 35, wherein the working electrode comprises carbon steel or stainless steel (each type optionally covered with a layer of conducting polymer), the reference electrode comprises stainless steel, carbon steel, or Hastelloy (each type optionally covered with a layer of conducting polymer), and the counter electrode comprises stainless steel, carbon steel, or Hastelloy (each type optionally covered with a layer of conducting polymer).
Aspect 37. An apparatus comprising: a test vessel; an electrochemical probe coupled to the test vessel; a potentiostat; and a computer operably connected to the electrochemical probe, wherein the computer has one or more processors and a memory having instructions stored thereon that cause the one or more processors to: determine a reference point in time at which a chemical was introduced into a field fluid; determine a sample polarization resistance value in a sample of the field fluid at a measurement point in time; input the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and determine, based on the calibration equation, the concentration of the chemical in the sample of the field fluid.
Aspect 38. The apparatus of Aspect 37, wherein the calibration time is a calibration time period between a test reference point in time and a calibration point in time for concentration/polarization resistance data point pairs from which the calibration equation is generated.
Aspect 39. The apparatus of Aspect 37, wherein the sample of the field fluid is obtained from an oil and gas system, a geothermal system, a carbon capture system, a carbon storage system, a hydrogen transport system, a water transport system, or a water treatment system.
Aspect 40. The apparatus of Aspect 39, wherein the sample is obtained from a pipeline, a process flow line, a valve, a separation vessel, a storage vessel, or a combination thereof.
Aspect 41. The apparatus of Aspect 37, wherein the chemical is a corrosion inhibitor, a scale inhibitor, a gas hydrate inhibitor, or a biocide.
Aspect 42. The apparatus of Aspect 37, wherein the sample polarization resistance value is generated using a linear polarization resistance technique, electrochemical impedance spectroscopy, or chronoamperometry.
Aspect 43. The apparatus of Aspect 37, wherein the field fluid comprises a brine.
Aspect 44. The apparatus of Aspect 37, wherein the field fluid comprises a brine and a hydrocarbon, wherein the sample polarization resistance value is determined from the brine.
Aspect 45. The apparatus of Aspect 37, wherein the sample polarization resistance value is determined using an electrochemical probe placed into the sample of the field fluid.
Aspect 46. The apparatus of Aspect 45, wherein the electrochemical probe comprises an array of electrochemical sensors.
Aspect 47. The apparatus of Aspect 45, wherein the electrochemical probe comprises a working electrode, a reference electrode, and a counter electrode.
Aspect 48. The apparatus of Aspect 47, wherein the working electrode comprises carbon steel or stainless steel (each type optionally covered with a layer of conducting polymer), the reference electrode comprises stainless steel, carbon steel, or Hastelloy (each type optionally covered with a layer of conducting polymer), and the counter electrode comprises stainless steel, carbon steel, or Hastelloy (each type optionally covered with a layer of conducting polymer).

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method comprising:
adding a chemical to each of a plurality of calibration fluids at a test reference point in time for each of the plurality of calibration fluids, wherein a concentration of the chemical in each of the plurality of calibration fluids is known and is different in each of the plurality of calibration fluids;
generating a polarization resistance curve versus time for each of the plurality of calibration fluids, wherein the polarization resistance curve for each of the plurality of calibration fluids starts at a common start time relative to the test reference point in time; and
generating a calibration equation based on a calibration polarization resistance value in each of the polarization resistance curves at a calibration point in time that occurs after the test reference point in time;
wherein the calibration equation calculates a concentration of the chemical in a sample of a field fluid when a sample polarization resistance value that was determined for the sample of the field fluid is input to the calibration equation.

2. The method of claim 1, further comprising:
determining a field reference point in time at which the chemical was introduced into a field fluid;
determining a sample polarization resistance value in a sample of the field fluid at a measurement point in time;
inputting the sample polarization resistance value into the calibration equation; and
determining, based on the calibration equation, a field concentration of the chemical in the sample of the field fluid.

3. The method of claim 2, wherein a first time period between the field reference point in time and the measurement point in time is equal to a second time period between the test reference point in time and the calibration point in time.

4. The method of claim 2, wherein the sample of the field fluid is obtained from an oil and gas system, a geothermal system, a carbon capture system, a carbon storage system, a hydrogen transport system, a water transport system, or a water treatment system.

5. The method of claim 4, further comprising:
introducing or not introducing, based on the field concentration, an additional amount of the chemical into the oil and gas system, the geothermal system, the carbon capture system, the carbon storage system, the hydrogen transport system, the water transport system, or the water treatment system.

6. The method of claim 4, wherein the sample is obtained from a pipeline, a process flow line, a valve, a separation vessel, a storage vessel, or a combination thereof.

7. The method of claim 1, wherein the chemical is a corrosion inhibitor, a scale inhibitor, a gas hydrate inhibitor, or a biocide.

8. The method of claim 1, wherein the calibration polarization resistance values and the sample polarization resistance value are generated using a linear polarization resistance technique, electrochemical impedance spectroscopy, or chronoamperometry.

9. The method of claim 1, wherein each of the plurality of calibration fluids comprises a brine, wherein the calibration polarization resistance values are generated from the brine.

10. The method of claim 1, wherein the sample of the field fluid comprises a brine and a hydrocarbon, wherein the sample polarization resistance value is generated from the brine.

11. The method of claim 1, wherein the polarization resistance curve is generated for each of the plurality of calibration fluids using an electrochemical probe placed into a water phase of each of the plurality of calibration fluids.

12. The method of claim 11, wherein the electrochemical probe comprises an array of electrochemical sensors.

13. The method of claim 11, wherein the electrochemical probe comprises a working electrode, a reference electrode, and a counter electrode.

14. An apparatus comprising:
a test vessel;
an electrochemical probe coupled to the test vessel;
a potentiostat; and
a computer operably connected to the electrochemical probe, wherein the computer has one or more processors and a memory having instructions stored thereon that cause the one or more processors to:
generate a polarization resistance curve versus time for each of a plurality of calibration fluids, wherein the polarization resistance curve for each of the plurality of calibration fluids starts at a common start time relative to a test reference point in time; and
generate a calibration equation based on a calibration polarization resistance value in each of the polarization resistance curves at a calibration point in time that occurs after the test reference point in time;
wherein the calibration equation calculates a concentration of a chemical in a sample of a field fluid when a sample polarization resistance value that was determined for the sample of the field fluid is input to the calibration equation.

15. The apparatus of claim 14, wherein the chemical is a corrosion inhibitor, a scale inhibitor, a gas hydrate inhibitor, or a biocide.

16. The apparatus of claim 14, wherein the calibration polarization resistance values and the sample polarization resistance value are generated using a linear polarization resistance technique, electrochemical impedance spectroscopy, or chronoamperometry.

17. The apparatus of claim 14, wherein each of the plurality of calibration fluids comprises a brine, wherein the calibration polarization resistance values are generated from the brine, wherein the sample of the field fluid comprises a brine and a hydrocarbon, wherein the sample polarization resistance value is generated from the brine.

18. The apparatus of claim 14, wherein the polarization resistance curve is generated for each of the plurality of calibration fluids using an electrochemical probe placed into a water phase of each of the plurality of calibration fluids.

19. A method comprising:
determining a reference point in time at which a chemical was introduced into a field fluid;
determining a sample polarization resistance value in a sample of the field fluid at a measurement point in time;
inputting the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and
determining, based on the calibration equation, the concentration of the chemical in the sample of the field fluid.

20. An apparatus comprising:
a test vessel;
an electrochemical probe coupled to the test vessel;
a potentiostat; and
a computer operably connected to the electrochemical probe, wherein the computer has one or more processors and a memory having instructions stored thereon that cause the one or more processors to:
determine a reference point in time at which a chemical was introduced into a field fluid;
determine a sample polarization resistance value in a sample of the field fluid at a measurement point in time;
input the sample polarization resistance value into a calibration equation that converts the sample polarization resistance value to a field concentration of the chemical in the sample of the field fluid, wherein the calibration equation is based on a calibration time that is equal to a time difference between the reference point in time and the measurement point in time; and
determine, based on the calibration equation, the concentration of the chemical in the sample of the field fluid.
